(19) United States
(12) Patent Application Publication
(10) Pub. No.: US 2007/0269541 A1
(43) Pub. Date: Nov. 22, 2007

(54) Method and compositions for relieving menopausal and perimenopausal symptoms

(76) Inventor: Peter Rohdewald, Munster (DE)

Correspondence Address: Eric Sinn, Esq.;Mintz, Levin, Cohn, Ferris,Glovsky and Popeo, P.C., The Chrysler Center, 666 Third Avenue, 24th Floor, New York, NY, 10017, US

(21) Appl. No.: 11/437,334

(22) Filed: May 19, 2006

(51) Int. Cl. A61K 36/87; A61K 36/13; A61K 31/7048; A61K 31/353

(52) U.S. Cl. 424/766; 424/770; 514/27; 514/456

(57) ABSTRACT

The present invention is directed to novel methods of treating and reducing perimenopausal and climacteric symptoms using compositions comprising proanthocyanidins. In one embodiment, the methods provide for perimenopausal and climacteric symptom reduction without the use of female hormones or hormone like substances.

### BACKGROUND

This invention relates to novel agents and novel methods for the treatment of perimenopausal and menopausal (climacteric) syndromes. Menopause occurs when the ovaries' production of estrogen begins to decline. As the body adapts to the reduced estrogen levels, menopausal (climacteric) syndromes becomes apparent. These symptoms may include hot flashes, palpitations, depression, anxiety, irritability, mood swings, lack of concentration, vaginal dryness, urgency of urination, and erratic menstrual periods.

Perimenopause is a period which is before actual menopause. At this time, the production of hormones such as estrogen and progesterone becomes irregular. During this period, the fertility of the female is significantly reduced. The perimenopausal period can last for a few months or for several years. Some clinicians maintain that perimenopause can last for as long as 5 to 15 years, while others refer to perimenopause as that period which is a 3 to 4 year span just before menopause. Either way, many women experience more symptoms during perimenopause than after menopause.

It is well known that climacteric symptoms and perimenopausal symptoms affect the quality of life of women and increases risk for cardiovascular events and osteoporosis. The decreased production of estrogen, associated with the perimenopausal and climacteric stages of life, is responsible for many undesirable physiological and psychic changes. Climacteric changes have been treated with a great variety of estrogens. For example, typical climacteric complaints, such as hot flashes and outbreaks of perspiration, insomnia, cardiovascular sensations, and sensations of dizziness are often treated by administration of synthetic estrogen (e.g., estradiol valerate). Psychic changes, manifesting themselves by emotional imbalance can likewise be eliminated by administration of synthetic estrogen. However, a disadvantage of using synthetic estrogen is that the treatment results in extensive proliferation of the endometrium, which leads, in turn, to undesirable uterine bleeding. The strong effect of estradiol valerate on the upper genital tract also limits the use of this compound. Furthermore, while estriol has a favorable effect on the lower genital tract (cervix uteri, vagina, and vulva), it has the disadvantage that typical complaints and psychic changes are not fully satisfactorily ameliorated. In addition, estrogen replacement therapy has been found to produce serious adverse effects as increased rate of cardiovascular diseases, thrombotic events and higher risk for breast and endometrial (genital tract) cancer. Bureau of Internal Affairs, Population Office (2002), Statistics of the Population, Web site of Population office, http://.ris.gov.tw/ch4/static/stI10-1-85-90; Li S, Holm K, Gulanick M, Lanuza D. Perimenopause and quality of life/commentary. Clinical Nursing Research 2000;9(1):6-26.

Because of these risks of existing therapy, there is a need to identify safe ways of enhancing quality of life of perimenopausal women, restoring the physiological and psychic balance as close as possible to normal.

One embodiment of the invention is directed to a method of reducing a climacteric symptom or a perimenopausal symptom in a female mammal - such as a human. The method comprises administering to the mammal an effective amount of a composition comprising proanthocyanidins. One preferred route of administration is oral administration. The composition may be, for example, a pill, a liquid extract, a food, a drink, or a beverage. The pill may be a capsule containing liquids or a solid pill. The liquid extract may be a concentrated extract. The food may be in the shape of a bar such as a chocolate (or other favor) bar, a semisolid such as a yogurt like substance, or an enriched food such as bread, rice, meat, gravy, cake and the like. The drink may be a health drink, or an enriched drink like based on a diary product (milk) or fruit juice. The beverage may be water, flavored water, soft drinks or an alcoholic drink.

The dosage that can be administered to a mammal may be between 10 mg per day to 6000 mg (6 grams) per day. In a preferred embodiment, the dosage is between 100 mg per day to 400 mg per day. In a more preferred embodiment, the dosage may be, for example, about 200 mg per day. The daily dosage described above may be split into multiple administrations such as, for example, two times a day, three times a day, or four times a day.

The composition comprising proanthocyanidins may be in the form of a plant material or may be obtained by synthesis (i.e., synthetic proanthocyanidins). For example, proanthocyanidins can be found in vegetable extracts, as well as in extracts of the bark of a maritime pine, the cones of cypresses, and the seeds and skin of grapes—an extract of each of these materials (i.e., pine bark extract, cypress cone extract, conifer extract, grape seed extract) would be suitable as a composition comprising proanthocyanidins.

In a preferred embodiment, the composition comprising proanthocyanidins may be a pine bark extract. The pine bark may be from P. pinaster, such as, for example, from Pycnogenol. In a preferred embodiment, the composition may contain proanthocyanidins at a concentration of 10% to 100% of total weight. For example, a Pycnogenol composition may be diluted or concentrated to contain 10%, 20%, 30%, 40%, 50%, 60%, 70%, 75%, 80%, 90% or 95% proanthocyanidins. Concentration may be performed using known methods such as column chromatography or affinity chromatography. Further, the Pycnogenol may be admixed with inactive ingredients to enhance solubility. These ingredients may include, for example, a protein or protein hydrolysates (e.g., degraded protein). The protein hydrolysate may have an average molecular weight of less than 5,000, less than 7,000, less than 10,000, less than 15,000, or less than 30,000 or a combination thereof. The combination thereof refers to a mixture of any two or more protein hydrolysates discussed above. For example, a mixture of protein hydrolysate of less than 7000 with a protein hydrolysate of less than 15,000. In one preferred embodiment, the protein or protein hydrolysate is collagen or partly degraded collagen.

In a preferred embodiment, the proanthocyanidins in the composition comprising proanthocyanidins is the sole active ingredient administered to the female mammal. That is, the female mammal is not treated with any external female hormone or hormone like substance by injection, by oral route, or by any method of administration. For example, the female mammal is not being treated with exogenous estrogen, phytoestrogen, and derivatives and functional analogs thereof of these hormones.

The methods of the invention are suitable for the treatment of female mammals that are in menopause—defined as not having a menstrual cycle for a period of one year. The method of the invention is also suitable for treatment of female mammals in the perimenopausal period. This period starts from approximately 15 years before menopause, 10 years before menopause, or 5 years before menopause in the typical woman. The average onset of menopause is about 50 years for a woman. Thus, the methods of the invention may be used for treating a woman who exhibits a perimenopausal symptom from the age of 35-50, 40-50 or 45-50.

The administration of the composition comprising may be for a period of 1 month, 2 months, 3 months, 6 months, or continuous (e.g., for as long as desired by the patient).

The symptoms to be treated, for any of the methods of the invention, include any and all the symptoms listed in Tables 5, 6, or 7. These undesirable symptoms include, at least, tiredness, headache, higher than normal urinary frequency, anxiety, vaginal dryness, menstrual problems, hot flashes, bloatedness, night sweat, backache, pain in limbs and depression. The treatment of a symptom include reducing the manifestation of the symptom, including, for example, reducing the frequency or severity of the symptom. Treatment may also include eliminating the symptom or delaying the onset of the symptom.

### BRIEF DESCRIPTION OF THE FIGURES

FIG. 1 depicts the frequency of symptoms for 155 perimenopausal women.

### DETAILED DESCRIPTION OF THE INVENTION

It is understood that the term “pine bark extract” in this disclosure refers to a French maritime pine bark extract which is, for example, commercially available as Pycnogenol® (Horphag). The terms “Pycnogenolg”, “pine bark extract” and “French maritime pine bark extract” are interchangeable in this disclosure.

Pinus pinaster (P. pinaster) and Pinus maritima (P. maritime), are understood to refer to the same organism commonly called “French Maritime Pine.” Hence, these terms are interchangeable.

Proanthocyanidins designates a group of flavonoids that includes the subgroups procyanidins, prodelphinidins and propelargonidins. Proanthocyanidins are homogeneous or heterogeneous polymers consisting of the monomer units catechin or epicatechin, which are connected either by 4-8 or 4-6 linkages, to the effect that a great number of isomer proanthocyanidins exist. Typically, the proanthocyanidins oligomers have a chain length of 2-12 monomer units. Proanthocyanidins may be synthesized or extracted from a plant material. Nonlimiting examples of plant material sources of proanthocyanidins include grape seeds, grape skin, pine barks, ginkgo leaves, peanuts, and cocoa beans, tamarind, tomato, peanut, almond, apple, cranberry, blueberry, tea leaves.

A well-known product containing proanthocyanidins, which is available in trade as a preparation of a food supplement under the name Pycnogenol®, is an extract of the maritime pine bark (Pinus pinaster). Pycnogenol®, the extract from French maritime pine bark (Pinus pinaster) is a registered trademark belonging to Horphag Research, Ltd. Pycnogenol® is a standardized bark extract of the French maritime pine Pinus pinaster, Aiton, subspecies Atlantica des Villar (Pycnogenol®, Horphag Research Ltd., UK). The quality of this extract is specified in the United States Pharmacopeia (USP 28) (Maritime Pine Extract. In: United States Pharmacopeia. Rockville: United States Pharmacopeial Convention, Inc.; 2005. pp. 2115-2116). The extract consists of a concentrate of polyphenols, which are also contained in fruits and vegetables, but, in low concentrations. The polyphenols are composed from flavonoids, especially procyanidins, and phenolic acids. All these constituents possess the ability to inactivate free radicals. Rohdewald P. A review of the French maritime pine bark extract (Pycnogenol®), a herbal medication with a diverse pharmacology. Int J Clin Pharmacol Ther 2002;40(4):158-168. Between 65-75% of Pycnogenol® are procyanidins comprising of catechin and epicatechin subunits with varying chain lengths (Rohdewald P. A review of the French maritime pine bark extract (Pycnogenol), an herbal medication with a diverse clinical pharmacology. Int J Clin Pharmacol Ther 2002;40: 158-168). Other constituents are polyphenolic monomers, phenolic or cinnamic acids and their glycosides (Id.).

Menopause is defined as a minimum of twelve months without menstruation.

Perimenopause refers to a period of a few months, to several years and up to 15 years before menopause. That is, perimenopause may occur in a woman between 45 to 50 years of age, between 40 to 50 years of age, or between 35 to 50 years of age.

We decided to investigate the potential benefits of Pycnogenol for perimenopausal women. The results of our study, and a discussion of the results are listed below in the Examples section.

### EXAMPLES

### Example 1

### Determining the Effectiveness in Using Pycnogenol for the Treatment of Climacteric or Perimenopausal Symptoms

As a basis of our study we evaluated on a group of 200 perimenopausal women in Taiwan the level of discomfort and the frequency of climacteric syndrome at enrollment. These values are compared with results obtained in other countries with perimenopausal women.

Patients and Methods

During a 3 and half years period (from January 2002 to July 2005), 200 perimenopausal women between 45-55 years old participated on the study.

Inclusion Criteria:

Menstrual cycles had disappeared for 3-11 months, but normal cycles appeared again. Patients were included according to hormone levels:

serum levels of FSH>30 IU/ml and estrogen E2 levels<20 pg/L. They were controlled for normal mammogram, endometrial thickness <6 mm, normal cervical smear (Pap-test).

Exclusion Criteria:

Systematic or acute diseases, hormone therapy, contraceptive medication, hormone substitution, oophrectomy, hysterectomy, illiteracy.

Participants were interviewed for socio-economical status, smoking and dietary habits and examined at first visit for BMI, heart rate, blood pressure, mammography, vaginal sonography and Pap test. Blood samples were taken for analysis with standard methods for total cholesterol, HDL, LDL, triglycerides, AST and ALT, FSH and estrogen levels were analyzed in samples taken on 3rd day of menstruation.

Total antioxidant status (TAS) was determined using a with Randox commercial kit, according to the method of Miller et al. Nicholas J. Miller, Catherine Rice-Evans, Michael J. Davies, Vimala Gopinathan, Anthony Milner. A novel method for measuring antioxidant capacity and its application to monitoring the antioxidant status in premature neonates. Clinical Science 1993; 84:407-412.

Patients visited clinic for screening, enrollment and 1, 3 and 6 months following start of treatment. At each visit, BMI, blood pressure, lipid profile and TAS were recorded, lipid profile was determined at start and after 3 and 6 month of treatment.

Medication

Patients received either 100 mg Pycnogenol® capsules or placebo twice daily over a period of 6 months. Capsules of Pycnogenol® and placebo, identical in shape and appearance, were prepared by Wide-Doctor, Int., Taiwan, and packaged with the same label. Patients were instructed to take the pills at breakfast and at dinner.

Compliance

During the first 3 months of treatment, researchers phoned each patient weekly to ensure compliance giving instructions about intake and questionnaire filling. From the 4^{th} month on, patients were phoned every two weeks until the end of the 6^{th} month.

Questionnaire

Women's Health Questionnaire, designed by Hunter (1992), was used by the subjects to describe level of discomfort and climacteric symptoms. Hunter M. The women's health questionnaire: a measure of physical and emotional well-being of mid-aged women. Psychology & Health 1992; 7(1):45-54. Patients delivered the filled forms at each visit to the investigators. Women's Health Questionnaire (WHQ) contains a total of 36 questions related to perimenopausal symptoms: 1. Somatic symptoms (7 items), 2. depressed mood (8 items), 3. vasomotor symptoms (2 items), 4. memory/concentration (3 items), 5 attractiveness (2 items), 6 anxiety (4 items), 7 sexual behavior (3 items), 8 sleep problems (3 items), 9 menstrual symptoms (4 items). Each symptom was evaluated according to its frequency of occurrence and discomfort level.

Points given for frequency: 4: never occurring; 3: sometimes occur; 2: frequently; 1 point: always occur. Points given for discomfort level: 4: no discomfort; 3: little discomfort; 2: clear discomfort; 1: heavy discomfort. The higher the score, the less pronounced was distress and dysfunction.

The reliability of the questionnaire has been tested before the start of our study. Item analysis of symptoms frequency was tested by calculating Cronbach's alpha coefficient, resulting in a high reliability of 0.94. For discomfort levels, Cronbach's alpha coefficient was 0.91, degree of re-testing was 0.85. After translation of the WHQ into Chinese we tested the translated questionnaire again for reliability and obtained a Cronbach's alpha coefficient of 0.899 for discomfort levels so that translation had no negative impact on the questionnaire.

Data Analysis

Data were analyzed by using SPSS (Statistical Package for the Social Science) for Windows 10.0 edition. Statistical analysis included descriptive statistics (age, and health conditions, other variables of the population) tested by paired t test. Differences in baseline performance between the two groups were tested with a one-way ANOVA test. A two-way ANOVA was performed with perimenopausal symptom scores obtained during treatment. Post-hoc comparisons were made with Sheffe's F-test. Significance was set at a probability value of <0.05.

Results

Pycnogenol® and placebo groups did not differ in respect to frequency of climacteric symptoms and severity of symptoms at enrollment; they were carefully matched in terms of age, body mass index, social-economic status and nicotine or caffeine consumption.

Statistical evaluation of placebo- and Pycnogenol® group (at this point, the group was designated to be treated with Pycnogenol but have not actually been administered any Pycnogenol) revealed no significant differences between groups (Table 1). Influences on antioxidant status—consumption of tea, fruits, vegetables—did not differ, nor differentiated between socio-economic status, BMI or blood pressure. BMI did not change significantly during treatment period.

Drop-Out Rate

From the 200 patients enrolled into the study, 175 patients completed the 6 months treatment period. From these 175 patients, only 155 completed all questionnaires and participated on all investigations, 80 patients in the Pycnogenol® group, 75 in the placebo group. The dropout rate was nearly the same for both groups and mainly caused by non-adherence to protocol (non compliance).

A total 45 patients dropped out from the study. 20 from the Pycnogenol® group and 25 from the placebo group. Among the 20 patients, one suffered from a car accident and one from pelvic infectious disease. From the 25 drop-outs in the placebo group, 2 moved from the city and one complained of weight gain during the study period. The other patients were excluded from evaluation because of lack of compliance. The drop-out rate was 22.5%. None of the patients terminated the study because of unwanted effects of treatment.

Results of blood pressure monitoring show a slight decrease of systolic and diastolic blood pressure for both groups without significant differences between groups (Table 2). In the lipid profile, triglyceride levels remained unaffected in both groups. HDL levels increased significantly relative to start in the Pycnogenol® group, however, difference to placebo was not significant. LDL values dropped significantly by 10% under Pycnogenol® treatment relative to start as well as compared to placebo (Table 2). These results indicate a lowering of the atherosclerotic index, i.e. the balance between HDL and LDL cholesterol, following treatment with Pycnogenol®.

During treatment with Pycnogenol® the total antioxidant status increased steadily and significantly relative to start. The values after 6 month of supplementation with Pycnogenol® were significantly higher compared to results of the placebo group (Table 2).

To get an overview about the most relevant climacteric symptoms of Taiwanese women, the means of the overall symptoms scores for 155 women from both groups before treatment had been calculated (Table 3). Both groups did not differ in severity of symptoms, expressed as symptom scores, at enrollment. At enrollment to the study, no statistically significant difference was observed between Pycnogenol®-group and placebo group in respect to frequency of symptoms, reported according in the WHQ, (Table 4).

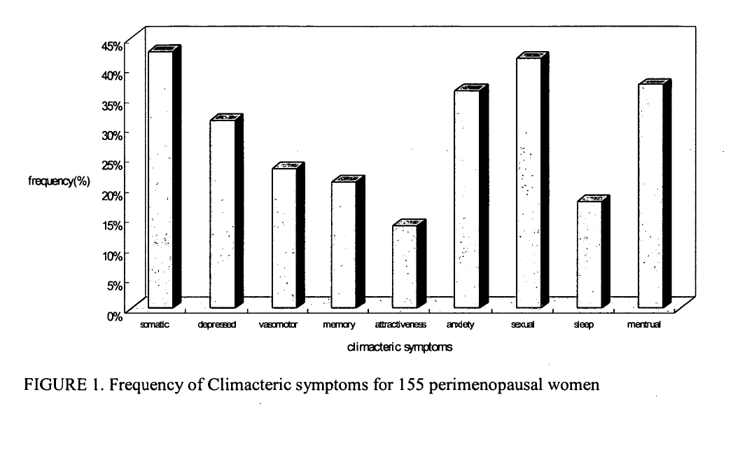

Variation between frequency of the different symptoms ranges from “not that frequently” (2.3) to “rarely occurring” (3.3). Most frequent symptoms were somatic symptoms such as tiredness, headache and urinary frequency, anxiety, followed by sexual (vaginal dryness)and menstrual problems, whereas vasomotor symptoms (hot flashes) were rarely reported (FIG. 1). About 25% of perimenopausal women complained frequently or sometimes of hot-flash. The comparison with frequency of symptoms in other countries shows some differences. Women in Italy complained during perimenopausal period most frequently of loss of memory, sleep disorder and vasomotor symptoms (hot flashes); Apolone G. Mosconi P. The Italian SF-36 Health Survey: translation, validation and norming. Journal of Clinical Epidemiology 1998; 51(11):1025-36. Data for women in England were similar. Wiklund I. Karlberg J. Lindgren R. Sandin K. Mattsson L A. A Swedish version of the Women's Health Questionnaire. A measure of postmenopausal complaints. Acta Obstetricia et Gynecologica Scandinavica 1993; 72(8):648-55.

During treatment, a rapid improvement of symptoms was reported from women in the Pycnogenol® group, starting after 1 month, Table 5. Only the symptom nausea did not change during treatment, whereas all other symptoms of the WHQ improved, in most cases statistically significant (p<0.01), compared to start of treatment. In the placebo group, no systematic statistically significant changes of symptoms were reported. Only occasionally symptoms improved after the first month of treatment significantly, however, later symptoms worsened, except for the question regarding poor memory. However, the increase in scores for the placebo group was clearly lower compared to the Pycnogenol® group.

The mean WHQ scores for the different categories of climacteric symptoms demonstrate the highly significant changes relative to start (p<0.01) in every category for each period of treatment with Pycnogenol® (Table 6). In most categories, placebo did not produce significant changes, except for memory and concentration and somatic symptoms. Sleep behavior and menstrual symptoms were significantly alleviated only at one point.

Pycnogenol® was evidently superior to placebo, especially in the categories attractiveness, sleep behavior, somatic problems and sexual problems (Table 6).

The difference in the frequency of symptoms at enrollment was not statistically significantly for both groups. Frequency of symptoms decreased continuously during treatment. After 6 months, the difference between Pycnogenol® group and placebo group became so clearly visible so that no statistical evaluation is required to demonstrate the advantage of Pycnogenol® treatment (Table 7). Whereas in the Pycnogenol® group reports for symptoms “always occur” and “sometimes occur” dropped down to zero, that frequency was still reported in the placebo group, at least as “sometimes occurring” after 6 months.

Patients did not report any undesirable side effects from the treatment with Pycnogenol®.

Discussion

The significant higher total antioxidant status (TAS) in blood of the Pycnogenol® group demonstrates that intake of Pycnogenol® increases indeed the antioxidant activity in blood. These results of cholesterol and TAS measurements suggest a positive, protective contribution of Pycnogenol® to vascular health.

Our evaluation of frequency of climacteric symptoms in perimenopausal women in Taiwan revealed somatic symptoms (tiredness, headache) as the most prominent symptoms, followed by anxiety and menstrual problems. Comparison with results obtained with the WHQ in other countries showed principal differences, as in Italian, Apolone G. Mosconi P. The Italian SF-36 Health Survey: translation, validation and norming. Journal of Clinical Epidemiology 1998; 51(11):1025-36, or English, Wiklund I. Karlberg J. Lindgren R. Sandin K. Mattsson L A. A Swedish version of the Women's Health Questionnaire. A measure of postmenopausal complaints. Acta Obstetricia et Gynecologica Scandinavica 1993; 72(8):648-55, women, the most important symptoms are vasomotor symptoms (hot-flash), loss of memory, attractiveness and sleep problems.

The results may lead one to suggest that Pycnogenol® could act perhaps as a phytoestrogen. Such hypothesis is unlikely for a number of reasons:

None of the known constituents of Pycnogenol®—catechin, taxifolin, phenolic acids, procyanidins is known to act as a phytoestrogen. These molecules do not show the linear arrangement of two hydroxy-group at the opposite ends of the molecule, which is characteristic for phytoestrogens.

Treatment of patients with endometriosis with Pycnogenol® improved symptoms, but did not alter estrogen levels (E2) during a treatment period of 48 weeks. Kohama T. Pycnogenol alleviates pain associated with pregnancy. Phytother Res 2006; in press. A phytoestrogen should lower estrogen levels by a feedback mechanism.

Furthermore, Pycnogenol® exerts positive effects on male erectile function, as demonstrated in a double blind, placebo-controlled study with patients suffering from erectile dysfunction. Durackova Z, Trebaticky B, Novotny V, Zitnanova A, Breza J. Lipid metabolism and erectile function improvement by Pycnogenol®, extract from the bark of Pinus pinaster in patients suffering from erectile dysfunction—a pilot study. Nutr Res 2003;23: 1189-1198. In cases of male infertility, supplementation with Pycnogenol® improved quality of sperms, Roseff S J. Improvement in Sperm Quality and Function with French Maritime Pine Tree Bark Extract. J Reprod Med 2002; 47:821-824, Stanislavov R, Nikolova V. Prelox® plus testosterone for achieve fertilization in previously infertile men. Eur Bull of Drug Research 2005; 13(l):7-13. In combination with L-arginine, Pycnogenol® normalized erectile function in 84% of men with erectile dysfunction, Stanislavov R, Nikolova V. Treatment of erectile dysfunction with Pycnogenol® and L-arginine. J Sex Marital Ther 2003; 29:207-213, Stanislavov R, Nikolova V. Prelox® plus testosterone for achieve fertilization in previously infertile men. Eur Bull of Drug Research 2005; 13(1):7-13. A phytoestrogen should show negative effects on erectile function. Thus, this is further evidence that Pycnogenol does not act as a phytoestrogen and is most probably not related to a hormonal effect.

In summary, the improvement of climacteric symptoms following 6 months supplementation with Pycnogenol® is very worthwhile, even when symptoms were only gradually improved, as frequency of symptoms was clearly reduced and, last not least, every category of symptoms was improved.

In the placebo group, a slight improvement of severity of symptoms and of frequency of symptoms was found, however, the changes relative to enrollment were minimum and seldom significant. The rather low placebo effect is very astonishing, when compared to the large placebo effects observed in studies with analgesics or antacids. This finding seems to demonstrate the reliability and robustness of the WHQ questionnaire.

In conclusion, perimenopausal symptoms according to the WHQ of Taiwanese women differ to some extent in frequency to reports from Europe. Antioxidant status and atherosclerotic index (ration LDL/HDL) were improved by Pycnogenol®. Supplementation with Pycnogenol® reduced clearly frequency of symptoms as well as severity of climacteric symptoms. As all symptoms were gradually improved without adverse effects, quality of life of perimenopausal women was ameliorated by Pycnogenol®.

All references, patents, and patent applications cited are hereby incorporated by reference in their entirety.

TABLE 1
BASELINE CHARACTERISTICS OF SUBJECTS

| | | Pycnogenol ® | Placebo | |
|---|---|---|---|---|
| | | (Mean ± SD) | (Mean ± SD) | |
| | | n = 80 | n = 75 | P value |
| Mean age (y) | | 46.73(5.09) | 47.02(4.22) | 0.728 |
| Height cm | | 156.04(5.00) | 155.15(5.76) | 0.340 |
| Weight kg | Enrollment | 58.27(8.16) | 57.50(7.28) | 0.477 |
| | After 6 | 57.97(8.61) | 57.32(7.25) | |
| | months | | | |
| | Mean change | −0.30 | −0.18 | |
| BMI (kg/m²) | Enrollment | 24.12(3.07) | 24.06(2.84) | 0.269 |
| | After 6 | 23.90(3.33) | 24.12(2.84) | |
| | months | | | |
| | Mean change | −0.22 | 0.06 | |
| Systolic blood pressure | | 116.37(14.10) | 116.38(15.10) | 0.996 |
| (mmHg) | | | | |
| Diastolic blood pressure | | 72.14(9.08) | 72.43(8.45) | 0.849 |
| (mmHg) | | | | |
| | | n(%) | n(%) | |
| Coffee intake | Every day | 5(7.2) | 4(6.5) | 0.784* |
| | >4 times/ | 0(0.0) | 2(3.2) | |
| | per week | | | |
| | 2-3 times/ | 13(18.8) | 12(19.4) | |
| | per week | | | |
| | <1 time/ | 34(49.3) | 29(46.8) | |
| | per week | | | |
| | None | 17(24.6) | 15(24.2) | |
| Tea intake | Every day | 17(25.8) | 18(30.5) | 0.909* |
| | >4 times/ | 8(12.1) | 8(13.6) | |
| | per week | | | |
| | 2-3 times/ | 26(39.4) | 14(23.7) | |
| | per week | | | |
| | <1 time/ | 11(16.7) | 13(22.0) | |
| | per week | | | |
| | None | 4(6.1) | 6(10.2) | |
| Smokers | % of group | 1(1.4) | 3(4.6) | 0.278 |
| Employee | % of group | 15(24.2) | 14(24.1) | 0.994 |
| Worker | % of group | 16(25.8) | 12(20.7) | 0.512 |

*The p values of 0.784 and 0.909 refer to differences of the coffee and tea drinking habits between the Pycnogenol ® and placebo group, indicating no group differences for caffeine consumption.

TABLE 2
BLOOD PRESSURE, LIPID PROFILE AND ANTIOXIDANT STATUS DURING
INTAKE OF PYCNOGENOL ® OR PLACEBO

| | Pycnogenol ® | | | | Placebo | | | |
|---|---|---|---|---|---|---|---|---|
| | Enrollment | 1 month | 3 months | 6 months | Enrollment | 1 month | 3 months | 6 months |
| Systolic blood | 116.37 | 113.41 | 110.27*** | 111.86** | 116.38 | 115.00 | 112.68** | 114.36 |
| Pressure | (14.10) | (14.78) | (12.75) | (14.09) | (15.10) | (12.53) | (13.68) | (11.65) |
| Diastolic blood | 72.14 | 70.18 | 69.37* | 69.58* | 72.43 | 71.02 | 69.68** | 71.71 |
| Pressure | (9.08) | (7.79) | (8.30) | (9.61) | (8.45) | (9.20) | (8.65) | (6.84) |
| HDL | 44.70 | — | 45.31** | 46.75* | 43.40 | — | 44.19 | 44.23 |
| | (10.40) | | (7.31) | (8.08) | (7.99) | | (7.54) | (9.63) |
| LDL | 111.44 | — | 105.27^{kk} | 100.41*^{kkk} | 120.12 | — | 126.34 | 121.85 |
| | (29.62) | | (25.16) | (24.24) | (38.01) | | (45.52) | (29.00) |
| Triglyceride | 90.37 | — | 99.32 | 94.46 | 113.13 | — | 114.44 | 114.09 |
| | (60.26) | | (48.45) | (55.04) | (76.59) | | (75.37) | (68.56) |
| TAS | 1.42 | 1.48** | 1.49** | 1.55***^{kk} | 1.41 | 1.45* | 1.44 | 1.43 |
| | (0.13) | (0.14) | (0.14) | (0.55) | (0.13) | (0.15) | (0.14) | (0.14) |

Mean difference to start is significant at the level 0.05*, 0.01**, 0.001***. Mean difference to placebo is significant at the level 0.01^{kk}, 0.001^{kkk}.
(Independent samples T-test)

BLOOD PRESSURE, LIPID PROFILE AND ANTIOXIDANT STATUS DURING
INTAKE OF PYCNOGENOL ® OR PLACEBO (ANOVA)

| | Pycnogenol ® | | | | | | Placebo | | | | | |
|---|---|---|---|---|---|---|---|---|---|---|---|---|
| | | | | | | Post | | | | | | Post |
| | | | | | | Hoc | | | | | | Hoc |
| | Enrollment | 1 month | 3 months | 6 months | p value | Tests | Enrollment | 1 month | 3 months | 6 months | p value | Tests |
| Systolic blood | 116.37 | 113.41 | 110.27 | 111.86 | 0.103 | | 116.38 | 115.00 | 112.68 | 114.36 | 0.552 | |
| Pressure | (14.10) | (14.78) | (12.75) | (14.09) | | | (15.10) | (12.53) | (13.88) | (11.65) | | |
| Diastolic blood | 72.14 | 70.18 | 69.37 | 69.58 | 0.267 | | 72.43 | 71.02 | 69.68 | 71.71 | 0.382 | |
| Pressure | (9.08) | (7.79) | (8.30) | (9.61) | | | (8.45) | (9.20) | (8.65) | (6.84) | | |
| HDL | 44.70 | — | 45.31 | 46.75 | 0.667 | | 43.40 | — | 44.19 | 44.23 | 0.949 | |
| | (10.40) | | (7.31) | (8.08) | | | (7.99) | | (7.54) | (9.63) | | |
| LDL | 111.44 | — | 105.27 | 100.41 | 0.206 | | 120.12 | — | 126.34 | 121.85 | 0.858 | |
| | (29.62) | | (25.16) | (24.24) | | | (38.01) | | (45.52) | (29.00) | | |
| Triglyceride | 90.37 | — | 99.32 | 94.46 | 0.981 | | 113.13 | — | 114.44 | 114.09 | 0.942 | |
| | (60.26) | | (48.45) | (55.04) | | | (76.59) | | (75.37) | (68.56) | | |
| TAS | 1.42 | 1.48 | 1.49 | 1.55 | 0.000 | 4 > 1 | 1.41 | 1.45 | 1.44 | 1.43 | 0.321 | |
| | (0.13) | (0.14) | (0.14) | (0.15) | | | (0.13) | (0.15) | (0.14) | (0.14) | | |

TWO-WAY ANOVA FOR BLOOD PRESSURE, TAS AND LIPID PROFILES
BETWEEN THE ANTIOXIDENT GROUP(AG) AND PLACEBO GROUP(PG).

| | group | timing | group * timing | Post Hoc Tests |
|---|---|---|---|---|
| Systolic blood pressure | 1.487 | 2.569 | 0.204 | |
| Diastolic blood pressure | 1.162 | 2.110 | 0.239 | |
| HDL | 2.720 | 0.766 | 0.206 | |
| LDL | 21.223*** | 0.645 | 1.461 | AG < PG |
| Triglyceride | 5.130* | 0.038 | 0.032 | AG < PG |
| TAS | 13.114*** | 5.335** | 2.503 | AG > PG t2, t4 > t1 |

*The mean difference is significant at the 0.05 level.
**The mean difference is significant at the 0.01 level.
***The mean difference is significant at the 0.001 level.

TABLE 3
CLIMACTERIC SYMPTOMS AT ENROLLMENT FOR BOTH
GROUPS

| | Pycnogenol ® group | Placebo group |
|---|---|---|
| | (n = 80) | (n = 75) |
| | Enrollment | Enrollment |
| Depressed | | |
| Miserable and sad | 3.15 | 3.11 |
| | (0.80) | (0.85) |
| Loss of interest in things | 2.97 | 3.08 |
| | (0.89) | (0.82) |
| Still enjoy the things | 2.91 | 2.98 |
| | (0.97) | (0.77) |
| Life not worth living | 3.28 | 3.17 |
| | (0.82) | (0.90) |
| Have a good appetite | 3.12 | 3.11 |
| | (0.87) | (0.86) |
| Irritability | 2.46 | 2.50 |
| | (0.75) | (0.69) |
| Worry about growing old | 2.50 | 2.36 |
| | (0.96) | (0.90) |
| Reduced well-being | 2.85 | 3.03 |
| | (0.85) | (0.91) |
| Sleep | | |
| Restlessness | 2.65 | 2.76 |
| | (0.85) | (0.82) |
| Early morning wakening | 2.50 | 2.52 |
| | (0.81) | (0.90) |
| Difficulty getting off to sleep | 2.59 | 2.41 |
| (0.98) | (0.98) | |
| Anxiety | | |
| Panicky feelings | 3.03 | 3.08 |
| | (0.86) | (0.60) |
| Anxiety leaving house alone | 3.19 | 3.31 |
| | (0.91) | (0.83) |
| Palpitation | 2.55 | 2.76 |
| | (0.83) | (0.84) |
| Feel tense/wound up | 2.60 | 2.78 |
| | (0.89) | (0.82) |
| Menstrual | | |
| Breast tenderness | 3.09 | 3.05 |
| | (0.85) | (0.76) |
| Abdominal cramps | 3.07 | 3.12 |
| | (0.84) | (0.74) |
| Heavy bleeding | 2.59 | 2.50 |
| | (0.96) | (0.95) |
| Bloatedness | 2.71 | 3.30 |
| | (0.99) | (0.46) |
| Vasomotor | | |
| Hot flashes | 3.37 | 3.20 |
| | (1.00) | (1.02) |
| Night sweats | 3.38 | 3.48 |
| | (0.91) | (0.73) |
| Somatic | | |
| Headaches | 2.20 | 2.63 |
| | (0.83) | (0.91) |
| Tiredness | 2.07 | 1.98 |
| | (0.74) | (0.80) |
| Dizzy | 2.57 | 2.40 |
| | (0.86) | (1.00 |
| Backache/pains in | 2.26 | 2.33 |
| limbs | (0.88) | (0.86) |
| Nausea | 3.36 | 3.23 |
| | (0.77) | (0.85) |
| Pain and needles in | 3.10 | 3.00 |
| hands & feet | (0.97) | (1.00) |
| Urinay frequency | 2.63 | 2.35 |
| | (0.99) | (0.98) |
| Sexual | | |
| Loss of sexual | 2.67 | 2.51 |
| interest | (0.85) | (0.81) |
| Dissatisfaction | 3.08 | 3.04 |
| | (0.76) | (0.68) |
| Vaginal dryness | 2.41 | 2.61 |
| | (0.93) | (0.84) |
| Attractiveness | | |
| Not lively | 2.18 | 2.39 |
| | (0.82) | (0.76) |
| Feeling unattractive | 2.35 | 2.50 |
| | (1.02) | (0.85) |
| Memory | | |
| Clumsiness | 2.88 | 3.10 |
| | (0.83) | (0.78) |
| Difficulty in | 2.36 | 2.61 |
| concentrating | (0.85) | (0.70) |
| Poor memory | 1.93 | 2.02 |
| | (0.82) | (0.83) |

TABLE 4
FREQUENCY OF SYMPTOMS FOR PERIMENOPAUSAL
WOMEN BEFORE ENROLLMENT (%)

| | Pycnogenol ® (n = 80) | | | | Placebo group(n = 75) | | | | |
|---|---|---|---|---|---|---|---|---|---|
| | definitely | sometimes | not much | not at all | definitely | sometimes | not much | not at all | p value |
| Miserable and sad | 1.5 | 20.6 | 39.7 | 38.2 | 4.6 | 16.9 | 41.5 | 36.9 | 0.783 |
| Loss of interest in thing | 6.0 | 22.4 | 40.3 | 31.3 | 3.1 | 20.0 | 43.1 | 33.8 | 0.473 |
| Still enjoy the things | 30.4 | 42.0 | 15.9 | 11.6 | 25.0 | 51.6 | 20.3 | 3.1 | 0.640 |
| Life not worth living | 2.9 | 14.5 | 34.8 | 47.8 | 3.1 | 23.4 | 26.6 | 46.9 | 0.489 |
| Have a good appetite | 39.1 | 37.7 | 18.8 | 4.3 | 39.1 | 35.9 | 21.9 | 3.1 | 0.965 |
| Irritability | 6.0 | 50.7 | 34.3 | 9.0 | 4.7 | 46.9 | 42.2 | 6.3 | 0.767 |
| Worry about growing old | 19.1 | 38.2 | 27.9 | 14.7 | 20.2 | 36.9 | 33.8 | 9.2 | 0.715 |
| Reduced well-being | 8.8 | 17.6 | 52.9 | 20.6 | 7.8 | 15.6 | 42.2 | 34.2 | 0.246 |
| Restlessness | 8.7 | 39.1 | 37.7 | 14.5 | 6.2 | 40.0 | 38.5 | 15.4 | 0.724 |
| Early morning wakening | 11.4 | 34.3 | 45.7 | 8.6 | 15.4 | 30.8 | 41.5 | 12.3 | 0.964 |
| Difficulty getting off to sleep | 13.8 | 36.9 | 27.7 | 21.5 | 21.9 | 32.8 | 31.3 | 14.1 | 0.264 |
| Panicky feelings | 2.9 | 26.5 | 35.3 | 35.3 | 3.1 | 18.5 | 46.2 | 32.3 | 0.742 |
| Anxiety leaving house alone | 6.0 | 14.9 | 32.8 | 46.3 | 3.1 | 14.1 | 31.3 | 51.6 | 0.439 |
| Palpitation | 5.8 | 44.9 | 33.3 | 15.9 | 4.7 | 42.2 | 34.4 | 17.2 | 0.592 |
| Feel tense/wound up | 9.0 | 40.3 | 32.8 | 17.9 | 6.3 | 46.9 | 29.7 | 17.2 | 0.901 |
| Breast tenderness | 1.5 | 26.9 | 32.8 | 38.8 | 6.2 | 26.2 | 38.5 | 29.2 | 0.233 |
| Abdominal cramps | 4.3 | 18.6 | 42.9 | 34.3 | 6.2 | 23.1 | 36.9 | 33.8 | 0.565 |
| Heavy bleeding | 19.6 | 33.3 | 33.3 | 13.7 | 18.0 | 28.0 | 50.0 | 14.0 | 0.645 |
| Bloatedness | 9.1 | 39.4 | 22.7 | 28.8 | 11.1 | 31.7 | 28.6 | 28.6 | 0.847 |
| Hot flashes | 9.0 | 14.9 | 25.4 | 50.7 | 7.8 | 25.0 | 20.3 | 46.9 | 0.510 |
| night sweats | 3.2 | 19.0 | 14.3 | 63.5 | 1.8 | 14.5 | 25.5 | 41.1 | 0.481 |
| Headaches | 14.7 | 48.5 | 27.9 | 8.8 | 15.9 | 31.7 | 39.7 | 12.7 | 0.232 |
| Tiredness | 17.6 | 63.2 | 13.2 | 5.9 | 29.2 | 46.2 | 21.5 | 3.1 | 0.507 |
| Dizzy | 7.1 | 45.7 | 30.0 | 17.1 | 23.1 | 27.7 | 21.5 | 3.1 | 0.286 |
| Backache/pains in limbs | 18.8 | 53.6 | 15.9 | 11.6 | 18.5 | 49.2 | 23.1 | 9.2 | 0.854 |
| Nausea | 1.4 | 13.0 | 33.3 | 52.2 | 3.1 | 17.2 | 32.7 | 46.9 | 0.363 |
| Pain and needles in hands & | 7.5 | 19.4 | 28.4 | 44.8 | 9.8 | 19.7 | 31.1 | 39.3 | 0.550 |
| feet | | | | | | | | | |
| Urinary frequency | 12.7 | 34.9 | 28.6 | 23.8 | 24.2 | 27.4 | 37.1 | 11.3 | 0.114 |
| Loss of sexual interest | 7.6 | 36.4 | 39.4 | 16.7 | 6.3 | 41.3 | 38.1 | 14.3 | 0.742 |
| Dissatisfaction | 1.9 | 19.2 | 48.1 | 30.8 | 1.9 | 15.1 | 60.4 | 22.6 | 0.781 |
| Vaginal dryness | 19.3 | 40.4 | 28.1 | 12.3 | 7.3 | 49.1 | 29.1 | 14.5 | 0.296 |
| Not lively | 19.4 | 49.3 | 25.4 | 6.0 | 17.2 | 39.1 | 37.5 | 6.3 | 0.304 |
| Feeling unattractive | 25.4 | 28.6 | 31.7 | 14.3 | 7.5 | 22.6 | 54.7 | 15.1 | 0.368 |
| Clumsiness | 3.0 | 31.8 | 39.4 | 25.8 | 7.7 | 29.2 | 40.0 | 23.1 | 0.533 |
| Difficulty in concentrating | 16.7 | 37.9 | 37.9 | 7.6 | 10.9 | 51.6 | 28.1 | 9.4 | 0.977 |
| Poor memory | 31.3 | 50.7 | 11.9 | 6.0 | 26.6 | 51.6 | 15.6 | 6.3 | 0.532 |

TABLE 5
CHANGE IN CLIMACTERIC SYMPTOMS BY WHQ SCORES BETWEEN
PYCNOGENOL ® AND PLACEBO GROUPS FROM ENROLLMENT
TO THE 1, 3, AND 6 MONTHS OF STUDY

| | Pycnogenol ® group(n = 80) | | | | | | | |
|---|---|---|---|---|---|---|---|---|
| | Enroll- | | | | placebo group(n = 75) | | | |
| | Ment | 1 month | 3 months | 6 months | Enrollment | 1 month | 3 months | 6 months |
| Depressed | | | | | | | | |
| Miserable and | 3.15 | 3.42** | 3.38* | 3.40* | 3.11 | 3.07 | 3.15 | 3.07 |
| sad | (0.80) | (0.58) | (0.53) | (0.49) | (0.85) | (0.90) | (0.79) | (0.89) |
| Loss of interest | 2.97 | 3.19 | 3.18 | 3.26 | 3.08 | 3.04 | 3.14 | 3.02 |
| in things | (0.89) | (0.66) | (0.44) | (0.44) | (0.82) | (0.86) | (0.82) | (0.81) |
| Still enjoy the | 2.91 | 3.28** | 3.17* | 3.38** | 2.98 | 2.98 | 2.78 | 2.85 |
| things | (0.97) | (0.70) | (0.56) | (0.49) | (0.77) | (0.97) | (0.92) | (0.91) |
| Life not worth | 3.28 | 3.30 | 3.46 | 3.45 | 3.17 | 3.14 | 3.11 | 3.28 |
| living | (0.82) | (0.78) | (0.54) | (0.50) | (0.90) | (0.84) | (0.91) | (0.86) |
| Have a good | 3.12 | 3.22 | 3.20 | 3.35 | 3.11 | 3.07 | 3.02 | 2.81 |
| appetite | (0.87) | (0.68) | (0.57) | (0.48) | (0.86) | (0.93) | (0.92) | (1.11) |
| Irritability | 2.46 | 2.95*** | 3.14*** | 3.12*** | 2.50 | 2.73 | 2.64 | 2.64 |
| | (0.75) | (0.65) | (0.35) | (0.32) | (0.69) | (0.75) | (0.67) | (0.73) |
| Worry about | 2.50 | 2.65 | 2.88* | 3.17*** | 2.36 | 2.62* | 2.43 | 2.52 |
| growing old | (0.96) | (0.94) | (0.73) | (0.62) | (0.90) | (0.91) | (0.77) | (0.77) |
| Reduced well- | 2.85 | 3.28*** | 3.28** | 3.23** | 3.03 | 3.25* | 3.04 | 2.95 |
| being | (0.85) | (0.64) | (0.58) | (0.43) | (0.91) | (0.76) | (0.92) | (0.86) |
| Sleep | | | | | | | | |
| Restlessness | 2.65 | 2.98** | 3.08*** | 3.16*** | 2.76 | 2.86 | 2.85 | 2.78 |
| | (0.85) | (0.53) | (0.49) | (0.62) | (0.82) | (0.75) | (0.72) | (0.96) |
| Early morning | 2.50 | 2.73* | 3.10*** | 3.14*** | 2.52 | 2.55 | 2.57 | 2.48 |
| wakening | (0.81) | (0.78) | (0.72) | (0.60) | (0.90) | (0.76) | (0.81) | (0.86) |
| Difficulty getting | 2.59 | 3.20*** | 3.48*** | 3.61*** | 2.41 | 2.58* | 2.50 | 2.41 |
| off to sleep | (0.98) | (0.79) | (0.60) | (0.74) | (0.98) | (0.99) | (1.01) | (0.86) |
| Anxiety | | | | | | | | |
| Panicky feelings | 3.03 | 3.37** | 3.29* | 3.30* | 3.08 | 2.88 | 2.96 | 3.10 |
| | (0.86) | (0.57) | (0.50) | (0.46) | (0.60) | (0.81) | (0.84) | (0.85) |
| Anxiety leaving | 3.19 | 3.31 | 3.43 | 3.47 | 3.31 | 3.16 | 3.11 | 3.07 |
| house alone | (0.91) | (0.76) | (0.58) | (0.50) | (0.83) | (0.88) | (0.88) | (0.93) |
| Palpitation | 2.55 | 3.11*** | 3.20*** | 3.17*** | 2.76 | 2.80 | 2.67 | 2.74 |
| | (0.83) | (0.64) | (0.51) | (0.61) | (0.84) | (0.85) | (0.79) | (0.84) |
| Feel tense/ | 2.60 | 3.08*** | 3.15*** | 3.14** | 2.78 | 2.64 | 2.61 | 2.58 |
| wound up | (0.89) | (0.67) | (0.46) | (0.35) | (0.82) | (0.88) | (0.79) | (0.85) |
| Menstrual | | | | | | | | |
| Breast tenderness | 3.09 | 3.27** | 3.33** | 3.19*** | 3.05 | 3.11 | 2.96 | 2.91 |
| | (0.85) | (0.62) | (0.55) | (0.39) | (0.76) | (0.79) | (0.79) | (0.90) |
| Abdominal cramps | 3.07 | 3.33* | 3.33 | 3.33 | 3.12 | 3.15 | 3.04 | 2.98 |
| | (0.84) | (0.57) | (0.55) | (0.48) | (0.74) | (0.78) | (0.81) | (0.91) |
| Heavy bleeding | 2.59 | 2.81 | 3.00 | 3.16* | 2.50 | 2.50 | 2.55 | 2.50 |
| | (0.96) | (0.82) | (0.48) | (0.37) | (0.95) | (0.85) | (0.93) | (0.90) |
| Bloatedness | 2.71 | 3.10*** | 3.12** | 3.30*** | 2.75 | 2.69 | 2.98 | 2.93 |
| | (0.99) | (0.62) | (0.48) | (0.46) | (1.00) | (0.94) | (0.87) | (0.91) |
| Vasomotor | | | | | | | | |
| Hot flashes | 3.37 | 3.63** | 3.68** | 3.86*** | 3.20 | 3.09 | 3.19 | 3.25 |
| | (1.00) | (0.69) | (0.57) | (0.60) | (1.02) | (0.89) | (0.77) | (0.74) |
| night sweats | 3.38 | 3.52 | 3.62 | 3.65 | 3.48 | 3.40 | 3.37 | 3.43 |
| | (0.91) | (0.67) | (0.53) | (0.48) | (0.73) | (0.81) | (0.74) | (0.68) |
| Somatic | | | | | | | | |
| Headaches | 2.20 | 2.94*** | 3.08*** | 3.13*** | 2.63 | 2.60 | 2.67 | 2.73 |
| | (0.83) | (0.72) | (0.65) | (0.58) | (0.91) | (0.53) | (0.83) | (0.90) |
| Tiredness | 2.07 | 2.89*** | 3.04*** | 3.04*** | 1.98 | 2.24* | 2.32* | 2.28 |
| | (0.74) | (0.69) | (0.50) | (0.39) | (0.80) | (0.74) | (0.73) | (0.70) |
| Dizzy | 2.57 | 2.92** | 3.08*** | 3.23*** | 2.40 | 2.70* | 2.68 | 2.71 |
| | (0.86) | (0.70) | (0.64) | (0.53) | (1.00) | (0.78) | (0.84) | (0.81) |
| Backache/pains in | 2.26 | 2.84*** | 3.00*** | 3.16*** | 2.33 | 2.43 | 2.43 | 2.28 |
| limbs | (0.88) | (0.71) | (0.66) | (0.62) | (0.86) | (0.85) | (0.74) | (0.68) |
| Nausea | 3.36 | 3.33 | 3.27 | 3.35 | 3.23 | 3.24 | 3.28 | 3.14 |
| | (0.77) | (0.72) | (0.53) | (0.48) | (0.85) | (0.74) | (0.71) | (0.87) |
| Pain and needles | 3.10 | 3.32 | 3.31 | 3.40* | 3.00 | 3.09 | 3.24 | 3.12 |
| In hands & feet | (0.97) | (0.64) | (0.58) | (0.50) | (1.00) | (0.82) | (0.80) | (0.80) |
| Urinary frequency | 2.63 | 3.10*** | 3.14*** | 3.12** | 2.35 | 2.48 | 2.60 | 2.62* |
| | (0.99) | (0.62) | (0.54) | (0.33) | (0.98) | (0.99) | (0.97) | (0.94) |
| Sexual | | | | | | | | |
| Loss of sexual | 2.67 | 2.98** | 3.23*** | 3.21*** | 2.51 | 2.69 | 2.75 | 2.65 |
| interest | (0.85) | (0.71) | (0.62) | (0.66) | (0.81) | (0.72) | (0.84) | (0.88) |
| Dissatisfaction | 3.08 | 3.20** | 3.31* | 3.29** | 3.04 | 2.92 | 3.17 | 3.29 |
| | (0.76) | (0.74) | (0.61) | (0.46) | (0.68) | (0.70) | (0.70) | (0.53) |
| Vaginal dryness | 2.41 | 2.92*** | 2.98*** | 3.18*** | 2.61 | 2.61 | 2.43 | 2.42 |
| | (0.93) | (0.74) | (0.62) | (0.64) | (0.84) | (0.83) | (0.83) | (0.82) |
| Attractiveness | | | | | | | | |
| Not lively | 2.18 | 2.69*** | 2.94*** | 3.05*** | 2.39 | 2.46 | 2.57 | 2.50 |
| | (0.82) | (0.85) | (0.51) | (0.22) | (0.76) | (0.75) | (0.70) | (0.85) |
| Feeling | 2.35 | 2.85*** | 3.02*** | 3.13*** | 2.50 | 2.77* | 2.82 | 2.61 |
| unattractive | (1.02) | (0.80) | (0.61) | (0.34) | (0.85) | (0.80) | (0.81) | (0.89) |
| Memory | | | | | | | | |
| Clumsiness | 2.88 | 3.03 | 3.12 | 3.09 | 3.10 | 2.96 | 2.88 | 2.78 |
| | (0.83) | (0.72) | (0.52) | (0.29) | (0.78) | (0.82) | (0.76) | (0.89) |
| Difficulty in | 2.36 | 2.78** | 3.06*** | 3.10*** | 2.61 | 2.72 | 2.51 | 2.36 |
| concentrating | (0.85) | (0.81) | (0.47) | (0.30) | (0.70) | (0.74) | (0.77) | (0.80) |
| Poor memory | 1.93 | 2.74*** | 2.92*** | 3.00*** | 2.02 | 2.22* | 2.23 | 2.33 |
| | (0.82) | (0.75) | (0.53) | (0.40) | (0.83) | (0.90) | (0.73) | (0.82) |

Values are presented as mean(S.D.)
*The mean difference is significant at the 0.05 level.
**The mean difference is significant at the 0.01 level.
***The mean difference is significant at the 0.001 level.
Differences are evaluated versus enrollment.

CHANGE IN CLIMACTERIC SYMPTOMS BY WHQ SCORES BETWEEN PYCNOGENOL ® AND
PLACEBO GROUPS FROM ENROLLMENT TO THE 1, 3, AND 6 MONTHS OF STUDY

| | Pycnogenol ® group(n = 80) | | | | | |
|---|---|---|---|---|---|---|
| | | | | | | Post Hoc |
| | Enrollment | 1 month | 3 months | 6 months | F | Tests |
| Miserable and sad | 3.15(0.80) | 3.42(0.58) | 3.38(0.53) | 3.40(0.49) | 2.552 | |
| Loss of interest in thing | 2.97(0.89) | 3.19(0.66) | 3.18(0.44) | 3.26(0.44) | 2.055 | |
| Still enjoy the things | 2.91(0.97) | 3.28(0.70) | 3.17(0.56) | 3.38(0.49) | 4.418** | T2.4 > 1 |
| Life not worth living | 3.28(0.82) | 3.30(0.78) | 3.46(0.54) | 3.45(0.50) | 1.065 | |
| Have a good appetite | 3.12(0.87) | 3.22(0.68) | 3.20(0.57) | 3.35(0.48) | 1.013 | |
| Irritability | 2.46(0.75) | 2.95(0.65) | 3.14(0.35) | 3.12(0.32) | 17.641*** | T2.3.4 > 1 |
| Worry about growing old | 2.50(0.96) | 2.65(0.94) | 2.88(0.73) | 3.17(0.62) | 9.631*** | t3 > 1, |
| | | | | | | t4 > 1.2 |
| Reduced well-being | 2.85(0.85) | 3.28(0.64) | 3.28(0.58) | 3.23(0.43) | 5.874** | T2.3.4 > 1 |
| Restlessness | 2.65(0.85) | 2.98(0.53) | 3.08(0.49) | 3.16(0.62) | 10.709*** | T2.3.4 > 1 |
| Early morning wakening | 2.50(0.81) | 2.73(0.78) | 3.10(0.72) | 3.14(0.60) | 11.604*** | t3 > 1.2, |
| | | | | | | t4 > 1.2 |
| Difficulty getting off to | 2.59(0.98) | 3.20(0.79) | 3.48(0.60) | 3.61(0.74) | 28.887*** | T2 > 1, t3 > 1, |
| sleep | | | | | | t4 > 1.2. |
| Panicky feelings | 3.03(0.86) | 3.37(0.57) | 3.29(0.50) | 3.30(0.46) | 3.540* | T2 > 1 |
| Anxiety leaving house | 3.19(0.91) | 3.31(0.76) | 3.43(0.58) | 3.47(0.50) | 1.822 | |
| alone | | | | | | |
| Palpitation | 2.55(0.83) | 3.11(0.64) | 3.20(0.51) | 3.17(0.61) | 15.849*** | T2.3.4 > 1 |
| Feel tense/wound up | 2.60(0.89) | 3.08(0.67) | 3.15(0.46) | 3.14(0.35) | 9.970*** | T2.3.4 > 1 |
| Breast tenderness | 3.09(0.85) | 3.27(0.62) | 3.33(0.55) | 3.19(0.39) | 1.546 | |
| Abdominal cramps | 3.07(0.84) | 3.33(0.57) | 3.33(0.55) | 3.33(0.48) | 2.628 | |
| Heavy bleeding | 2.59(0.96) | 2.81(0.82) | 3.00(0.48) | 3.16(0.37) | 4.433** | T4 > 1 |
| Bloatedness | 2.71(0.99) | 3.10(0.62) | 3.12(0.48) | 3.30(0.46) | 7.126*** | t2.3.4 > 1 |
| Hot flashes | 3.37(1.00) | 3.63(0.69) | 3.68(0.57) | 3.86(0.60) | 10.889 | t2.3.4 > 1 |
| night sweats | 3.38(0.91) | 3.52(0.67) | 3.62(0.53) | 3.65(0.48) | 1.713 | |
| Headaches | 2.20(0.83) | 2.94(0.72) | 3.08(0.65) | 3.13(0.58) | 20.583*** | t2.3.4 > 1 |
| Tiredness | 2.07(0.74) | 2.89(0.69) | 3.04(0.50) | 3.04(0.39) | 43.831*** | t2.3.4 > 1 |
| Dizzy | 2.57(0.86) | 2.92(0.70) | 3.08(0.64) | 3.23(0.53) | 10.463*** | t2.3.4 > 1 |
| Backache/pains in limbs | 2.26(0.88) | 2.84(0.71) | 3.00(0.66) | 3.16(0.62) | 23.428*** | t2.3.4 > 1 |
| Nausea | 3.36(0.77) | 3.33(0.72) | 3.27(0.53) | 3.35(0.48) | 0.189 | |
| Pain and needles in hands & | 3.10(0.97) | 3.32(0.64) | 3.31(0.58) | 3.40(0.50) | 1.835 | |
| feet | | | | | | |
| Urinary frequency | 2.63(0.99) | 3.10(0.62) | 3.14(0.54) | 3.12(0.33) | 7.214*** | t2.3.4 > 1 |
| Loss of sexual interest | 2.67(0.85) | 2.98(0.71) | 3.23(0.62) | 3.21(0.66) | 9.507*** | t2.3.4 > 1 |
| Dissatisfaction | 3.08(0.76) | 3.20(0.74) | 3.31(0.61) | 3.29(0.46) | 1.128 | |
| Vaginal dryness | 2.41(0.93) | 2.92(0.74) | 2.98(0.62) | 3.18(0.64) | 12.728*** | t2.3.4 > 1 |
| Not lively | 2.18(0.82) | 2.69(0.85) | 2.94(0.51) | 3.05(0.22) | 18.114*** | t2.3.4 > 1 |
| Feeling unattractive | 2.35(1.02) | 2.85(0.80) | 3.02(0.61) | 3.13(0.34) | 10.769*** | t2.3.4 > 1 |
| Clumsiness | 2.88(0.83) | 3.03(0.72) | 3.12(0.52) | 3.09(0.29) | 1.583 | |
| Difficulty in concentrating | 2.36(0.85) | 2.78(0.81) | 3.06(0.47) | 3.10(0.30) | 13.610*** | t2.3.4 > 1 |
| Poor memory | 1.93(0.82) | 2.74(0.75) | 2.92(0.53) | 3.00(0.40) | 33.868*** | t2.3.4 > 1 |

| | Placebo group(n = 75) | | | | | |
|---|---|---|---|---|---|---|
| | | | | | | Post |
| | | | | | | Hoc |
| | Enrollment | 1 month | 3 months | 6 months | F | Tests |
| Miserable and sad | 3.11(0.85) | 3.07(0.90) | 3.15(0.79) | 3.07(0.89) | 0.092 | |
| Loss of interest in thing | 3.08(0.82) | 3.04(0.86) | 3.14(0.82) | 3.02(0.81) | 0.169 | |
| Still enjoy the things | 2.98(0.77) | 2.98(0.97) | 2.78(0.92) | 2.85(0.91) | 0.641 | |
| Life not worth living | 3.17(0.90) | 3.14(0.84) | 3.11(0.91) | 3.28(0.86) | 0.297 | |
| Have a good appetite | 3.11(0.86) | 3.07(0.93) | 3.02(0.92) | 2.81(1.11) | 0.939 | |
| Irritability | 2.50(0.69) | 2.73(0.75) | 2.64(0.67) | 2.64(0.73) | 1.098 | |
| Worry about growing old | 2.36(0.90) | 2.62(0.91) | 2.43(0.77) | 2.52(0.77) | 1.291 | |
| Reduced well-being | 3.03(0.91) | 3.25(0.76) | 3.04(0.92) | 2.95(0.86) | 1.040 | |
| Restlessness | 2.76(0.82) | 2.86(0.75) | 2.85(0.72) | 2.78(0.96) | 1.012 | |
| Early morning wakening | 2.52(0.90) | 2.55(0.76) | 2.57(0.81) | 2.48(0.86) | 0.114 | |
| Difficulty getting off to | 2.41(0.98) | 2.58(0.99) | 2.50(1.01) | 2.41(0.86) | 0.525 | |
| sleep | | | | | | |
| Panicky feelings | 3.08(0.60) | 2.88(0.81) | 2.96(0.84) | 3.10(0.85) | 0.854 | |
| Anxiety leaving house | 3.31(0.83) | 3.16(0.88) | 3.11(0.88) | 3.07(0.93) | 0.789 | |
| alone | | | | | | |
| Palpitation | 2.76(0.84) | 2.80(0.85) | 2.67(0.79) | 2.74(0.84) | 0.321 | |
| Feel tense/wound up | 2.78(0.82) | 2.64(0.88) | 2.61(0.79) | 2.58(0.85) | 0.524 | |
| Breast tenderness | 3.05(0.76) | 3.11(0.79) | 2.96(0.79) | 2.91(0.90) | 0.622 | |
| Abdominal cramps | 3.12(0.74) | 3.15(0.78) | 3.04(0.81) | 2.98(0.91) | 0.454 | |
| Heavy bleeding | 2.50(0.95) | 2.50(0.85) | 2.55(0.93) | 2.50(0.90) | 0.031 | |
| Bloatedness | 2.75(1.00) | 2.69(0.94) | 2.98(0.87) | 2.93(0.91) | 1.105 | |
| Hot flashes | 3.20(1.02) | 3.09(0.89) | 3.19(0.77) | 3.25(0.74) | 0.451 | |
| night sweats | 3.48(0.73) | 3.40(0.81) | 3.37(0.74) | 3.43(0.68) | 0.241 | |
| Headaches | 2.63(0.91) | 2.60(0.53) | 2.67(0.83) | 2.73(0.90) | 0.685 | |
| Tiredness | 1.98(0.80) | 2.24(0.74) | 2.32(0.73) | 2.28(0.70) | 2.361 | |
| Dizzy | 2.40(1.00) | 2.70(0.78) | 2.68(0.84) | 2.71(0.81) | 1.706 | |
| Backache/pains in limbs | 2.33(0.86) | 2.43(0.85) | 2.43(0.74) | 2.28(0.68) | 0.897 | |
| Nausea | 3.23(0.85) | 3.24(0.74) | 3.28(0.71) | 3.14(0.87) | 0.220 | |
| Pain and needles in hands & | 3.00(1.00) | 3.09(0.82) | 3.24(0.80) | 3.12(0.80) | 0.691 | |
| feet | | | | | | |
| Urinary frequency | 2.35(0.98) | 2.48(0.99) | 2.60(0.97) | 2.62(0.94) | 0.831 | |
| Loss of sexual interest | 2.51(0.81) | 2.69(0.72) | 2.75(0.84) | 2.65(0.88) | 0.519 | |
| Dissatisfaction | 3.04(0.68) | 2.92(0.70) | 3.17(0.70) | 3.29(0.53) | 2.302 | |
| Vaginal dryness | 2.61(0.84) | 2.61(0.83) | 2.43(0.83) | 2.42(0.82) | 0.515 | |
| Not lively | 2.39(0.76) | 2.46(0.75) | 2.57(0.70) | 2.50(0.85) | 0.902 | |
| Feeling unattractive | 2.50(0.85) | 2.77(0.80) | 2.82(0.81) | 2.61(0.89) | 1.666 | |
| Clumsiness | 3.10(0.78) | 2.96(0.82) | 2.88(0.76) | 2.78(0.89) | 1.304 | |
| Difficulty in concentrating | 2.61(0.70) | 2.72(0.74) | 2.51(0.77) | 2.36(0.80) | 2.247 | |
| Poor memory | 2.02(0.83) | 2.22(0.90) | 2.23(0.73) | 2.33(0.82) | 1.444 | |

Values are presented as mean(SD)
*The mean difference is significant at the 0.05 level.
**The mean difference is significant at the 0.01 level.
***The mean difference is significant at the 0.001 level.

TABLE 6
MEAN CHANGE OF THE CLIMACTERIC SYMPTOMS EVALUATED BY THE WHQ
SCALE

| | Pycnogenol ® group(n = 80) | | | | placebo group(n = 75) | | | |
|---|---|---|---|---|---|---|---|---|
| | Enrollment | 1 month | 3 months | 6 months | Enrollment | 1 month | 3 months | 6 months |
| Somatic | 2.61 | 3.05⁼⁼⁼ | 3.14⁼⁼⁼ | 3.21⁼⁼⁼ | 2.57 | 2.69*** | 2.75** | 2.69 |
| problems | (0.97) | (0.69) | (0.50) | (0.41) | (1.00) | (0.89) | (0.87) | (0.87) |
| Depressed | 2.89 | 3.16⁼⁼ | 3.21⁼⁼⁼ | 3.29⁼⁼⁼ | 2.91 | 2.99* | 2.91 | 2.89 |
| | (0.91) | (0.74) | (0.54) | (0.46) | (0.89) | (0.88) | (0.87) | (0.89) |
| Vasomotor | 3.28 | 3.57⁼⁼ | 3.64⁼⁼⁼ | 3.76**⁼⁼⁼ | 3.27 | 3.25 | 3.28 | 3.34 |
| problems | (0.96) | (0.59) | (0.50) | (0.43) | (0.91) | (0.86) | (0.76) | (0.71) |
| Memory/ | 2.39 | 2.85⁼⁼⁼ | 3.03⁼⁼⁼ | 3.06⁼⁼⁼ | 2.39 | 2.54* | 2.64* | 2.67* |
| Concentration | (0.92) | (0.77) | (0.51) | (0.25) | (0.90) | (0.85) | (0.82) | (0.82) |
| Attractiveness | 2.26 | 2.77 | 2.98⁼⁼ | 3.09⁼⁼⁼ | 2.41 | 2.58 | 2.63 | 2.59 |
| | (0.92) | (0.82) | (0.56) | (0.28) | (0.85) | (0.80) | (0.80) | (0.80) |
| Anxiety | 2.85 | 3.22⁼⁼⁼ | 3.27⁼⁼⁼ | 3.27⁼⁼⁼ | 2.91 | 2.86 | 2.84 | 2.92 |
| | (0.91) | (0.58) | (0.50) | (0.44) | (0.88) | (0.85) | (0.87) | (0.88) |
| Sexual behavior | 2.67 | 3.04⁼⁼ | 3.18⁼⁼⁼ | 3.23⁼⁼⁼ | 2.71 | 2.76 | 2.81 | 2.79 |
| | (0.90) | (0.72) | (0.57) | (0.42) | (0.81) | (0.77) | (0.84) | (0.83) |
| Sleep | 2.55 | 2.98⁼⁼⁼ | 3.22⁼⁼⁼ | 3.31⁼⁼⁼ | 2.51 | 2.67** | 2.64 | 2.56 |
| | (0.88) | (0.63) | (0.50) | (0.47) | (0.91) | (0.85) | (0.86) | (0.90) |
| Menstrual | 2.89 | 3.15⁼⁼⁼ | 3.21⁼⁼ | 3.25⁼⁼⁼ | 2.80 | 2.81 | 2.96** | 2.92 |
| problems | (0.93) | (0.68) | (0.53) | (0.44) | (0.95) | (0.87) | (0.87) | (0.85) |

All means of symptoms were stat. sign. different to enrollment for the Pycnogenol ®-group (p > 0.001).
Significant differences to enrollment for the placebo group are indicated as p < 0.05*, p < 0.01**, p < 0.001***
Significant differences to placebo are indicated by ⁼⁼p < 0.01, ⁼⁼⁼p < 0.001.

TABLE 7
FREQUENCY OF ALWAYS OCCURRING SYMPTOMS AT
ENROLLMENT AND AFTER 6 MONTHS TREATMENT (%)

| | |
|---|---|
| Pycnogenol ® | Placebo |
| group | group |
| (months) | (months) |

| Symptom | 0 | 6 | 0 | 6 |
|---|---|---|---|---|
| Depressed | | | | |
| Miserable and sad | 1.5 | 0 | 4.6 | 7.1 |
| Loss of interest in thing | 6.0 | 0 | 3.1 | 2.4 |
| Still enjoy the things | 30.4 | 38.1 | 25.0 | 24.4 |
| Life not worth living | 2.9 | 0 | 3.1 | 2.6 |
| Have a good appetite | 39.1 | 34.9 | 39.1 | 33.3 |
| Irritability | 6.0 | 0 | 4.7 | 4.8 |
| Worry about growing old | 19.1 | 0 | 20.2 | 7.1 |
| Reduced well being | 8.8 | 0 | 7.8 | 7.3 |
| Sleep | | | | |
| Restlessness | 8.7 | 0 | 6.2 | 9.8 |
| Early morning wakening | 11.4 | 0 | 15.4 | 14.3 |
| Difficulty getting off to | 13.8 | 0 | 21.9 | 19.0 |
| sleep | | | | |
| Anxiety | | | | |
| Panicky feelings | 2.9 | 0 | 3.1 | 2.4 |
| Anxiety leaving house | 6.0 | 0 | 3.1 | 4.9 |
| alone | | | | |
| Palpitation | 5.8 | 0 | 4.7 | 7.1 |
| Feel tense/wound up | 9.0 | 0 | 6.3 | 2.4 |
| Menstrual | | | | |
| Breast tenderness | 1.5 | 0 | 6.2 | 0 |
| Abdominal cramps | 4.3 | 0 | 6.2 | 0 |
| Heavy bleeding | 19.6 | 16.1 | 18.0 | 14.7 |
| Bloatedness | 9.1 | 0 | 11.1 | 7.3 |
| Vasomotor | | | | |
| Hot flashes | 9.0 | 0 | 7.8 | 0 |
| Night sweats | 3.2 | 0 | 1.8 | 0 |
| Somatic | | | | |
| Headaches | 14.7 | 0 | 15.9 | 7.1 |
| Tiredness | 17.6 | 0 | 29.2 | 11.6 |
| Dizzy | 7.1 | 0 | 23.1 | 9.8 |
| Backache/pains in limbs | 18.8 | 0 | 18.5 | 12.5 |
| Nausea | 1.4 | 0 | 3.1 | 7.1 |
| Pain and needles in | 7.5 | 0 | 9.8 | 4.8 |
| hands & feet | | | | |
| Urinary frequency | 12.7 | 0 | 24.2 | 11.9 |
| Sexual | | | | |
| Loss of sexual interest | 7.6 | 0 | 6.3 | 5.3 |
| Dissatisfaction | 1.9 | 0 | 1.9 | 0 |
| Vaginal dryness | 19.3 | 0 | 7.3 | 8.6 |
| Attractiveness | | | | |
| Not lively | 19.4 | 0 | 17.2 | 7.1 |
| Feeling unattractive | 25.4 | 0 | 7.5 | 14.6 |
| Memory | | | | |
| Clumsiness | 3.0 | 0 | 7.7 | 0 |
| Difficulty in | 16.7 | 0 | 10.9 | 2.4 |
| concentrating | | | | |
| Poor memory | 31.3 | 0 | 26.6 | 11.9 |

1. A method of reducing treating or reducing symptoms associated with a climacteric symptom or a perimenopausal symptom in a female mammal comprising
(a) identifying a female mammal in menopause or perimenopause with a climacteric symptom or a perimenopausal symptom
(b) administering to the mammal an effective amount of a composition comprising proanthocyanidins.

2. The method of claim 1 wherein the mammal is a human.

3. The method of claim 1 wherein the composition comprising proanthocyanidins is administered orally.

4. The method of claim 1 wherein the composition comprising proanthocyanidins is administered at a dosage of between about 10 mg per day to about 6000 mg per day.

5. The method of claim 3 wherein the composition comprising proanthocyanidins is administered at a dosage of between about 100 mg per day to about 400 mg per day.

6. The method of claim 3 wherein the composition comprising proanthocyanidins is administered at a dosage of about 200 mg per day.

7. The method of claim 1 wherein the composition comprising proanthocyanidins is from a plant material or from a synthesized material.

8. The method of claim 7 wherein the plant material is a plant extract.

9. The method of claim 8 wherein the plant extract is a pine bark extract or a grape seed extract.

10. The method of claim 1 wherein the composition contains proanthocyanidins comprises proanthocyanidins at a concentration of between 10% to 100% of total weight.

11. The method of claim 1 in wherein the composition comprising proanthocyanidins is incorporated into a food.

12. The method of claim 1 wherein said proanthocyanidins is the sole active ingredient administered to said female mammal

13. The method of claim 1, wherein said female mammal is not treated with any external female hormones.

14. The method of claim 1, wherein said female mammal is not treated with estrogen or phytoestrogen.

15. The method of claim 1 wherein said perimenopausal symptom occurs from 15 years before menopause.

16. The method of claim 1 wherein said perimenopausal symptom occurs 10 years to menopause.

17. The method of claim 1 wherein said perimenopausal symptom occurs 5 years to menopause.

18. The method of claim 1, wherein administering comprises administering said composition to the patient for at least 1 month.

19. The method of claim 1, wherein administering comprises administering said composition to the patient for at least 6 months.
